(19) United States
(12) Patent Application Publication
(10) Pub. No.: US 2018/0051026 A1
(43) Pub. Date: Feb. 22, 2018

(54) POLYMORPHS OF IBRUTINIB

(71) Applicant: DR. REDDY'S LABORATORIES LIMITED, Telangana, Hyderabad (IN)

(72) Inventors: Vishweshwar PEDDY, Hyderabad (IN); Dharma Jagannadha Rao VELAGA, Vishakapatnam (IN); Sundara Lakshmi KANNIAH, Vellore (IN); Ramanaiah CHENNURU, Nellore (IN); Srividya RAMAKRISHNAN, Hyderabad (IN); Srinivas RANGINENI, Mahabubnagar (IN)

(21) Appl. No.: 15/553,559

(22) PCT Filed: Mar. 2, 2016
(86) PCT No.: PCT/IB2016/051164

§ 371 Date: Aug. 24, 2017

(30) Foreign Application Priority Data
Mar. 3, 2015 (IN) 1025/CHE/2015
Jul. 2, 2015 (IN) 3393/CHE/2015

(51) Int. Cl. C07D 487/04

(52) U.S. Cl. CPC C07D 487/04; C07B 2200/13

(57) ABSTRACT

The present invention provides crystalline forms of Ibrutinib and processes for their preparation.

### INTRODUCTION

The present invention provides polymorphs of Ibrutinib and process for their preparation.

### BACKGROUND OF THE INVENTION

The drug compound having the adopted name “Ibrutinib” has a chemical name I-((R)-3-(4-amino-3-(4-phenoxyphenyl)-IH-pyrazolo[3,4-d]pyrimidin-1-yl)piperidin-I-yl)prop-2-en-I-one, and is structurally represented below.

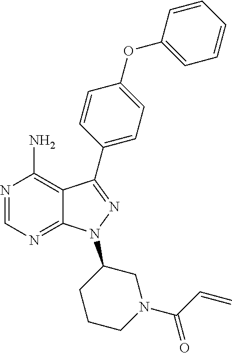 [SMILES: C=CC(=O)N1CCC[C@@H](n2nc(-c3ccc(Oc4ccccc4)cc3)c3c(N)ncnc32)C1]

Ibrutinib is an inhibitor of Bruton's tyrosine kinase (BTK) and is approved in US for the treatment of patients with mantle cell lymphoma and chronic lymphocytic leukemia who have received at least one prior therapy.

U.S. Pat. No. 7,514,444 discloses process for the preparation of Ibrutinib. The US '444 discloses isolation of Ibrutinib by flash chromatography using dichloromethane and methanol as eluents.

WO 2013/184572A1 application discloses crystalline, solvates and amorphous form of Ibrutinib. In particular, the application discloses polymorphic Forms A, B, C, D, E and F characterized by PXRD, IR, DSC and TGA. The WO '572 application discloses process for the preparation of amorphous form of Ibrutinib by dissolving Form A in dichloromethane. The solvent dichloromethane was removed under rotary evaporation to provide amorphous Ibrutinib.

CN103694241A discloses crystal form A of Ibrutinib characterized by PXRD. CN103923084A discloses crystal forms II, III, IV, V, VI, VII and VIII of Ibrutinib, characterized by PXRD pattern.

WO 2015/145415A2 application discloses various solid forms of Ibrutinib designated as Form III, Form IV, Form V, Form VI, Form VII, Form VIII and Form IX.

WO 2016/022942A1 application discloses solid dispersions of ibrutinib.

WO 2016/025720A1 application discloses crystalline forms of Ibrutinib designated as Form G, Form J and Form K.

The existence and possible numbers of polymorphic forms for a given compound cannot be predicted, and there are no “standard” procedures that can be used to prepare polymorphic forms of a substance. This is well-known in the art, as reported, for example, by A. Goho, “Tricky Business,” Science News, Vol. 166(8), August 2004.

There remains a need for alternate polymorphic forms of Ibrutinib and processes for preparing them.

### SUMMARY

The present invention provides crystalline forms of Ibrutinib designated as Form D1, Form D1a, Form D2, Form D2a, Form D3, Form D4, Form D5, Form D6, Form D7, Form D8, Form D9, Form D10, Form D11, Form D12 and Form D13 and processes for their preparation.

### BRIEF DESCRIPTION OF THE DRAWINGS

FIG. 1 illustrates a characteristic PXRD pattern of crystalline Ibrutinib Form D1 as obtained in example 1.

FIG. 2 illustrates a characteristic PXRD pattern of crystalline Ibrutinib Form D1 as obtained in example 2.

FIG. 3 illustrates a characteristic PXRD pattern of crystalline Ibrutinib Form D2 as obtained in example 3.

FIG. 4 illustrates a characteristic PXRD pattern of crystalline Ibrutinib Form D2 as obtained in example 4.

FIG. 5 illustrates a characteristic PXRD pattern of crystalline Ibrutinib Form D2a.

FIG. 6 illustrates a characteristic PXRD pattern of crystalline Ibrutinib Form D3 as obtained in example 8.

FIG. 7 illustrates a characteristic PXRD pattern of crystalline Ibrutinib Form D4 as obtained in example 9.

FIG. 8 illustrates a characteristic PXRD pattern of crystalline Ibrutinib Form D5 as obtained in example 10.

FIG. 9 illustrates a characteristic PXRD pattern of crystalline Ibrutinib Form D6 as obtained in example 11.

FIG. 10 illustrates a characteristic PXRD pattern of crystalline Ibrutinib Form D7 as obtained in example 12.

FIG. 11 illustrates a characteristic PXRD pattern of crystalline Ibrutinib Form D8 as obtained in example 13.

FIG. 12 illustrates a characteristic PXRD pattern of crystalline Ibrutinib Form D9 as obtained in example 16 and 17.

FIG. 13 illustrates a characteristic PXRD pattern of crystalline Ibrutinib Form D10 as obtained in example 18.

FIG. 14 illustrates a characteristic PXRD pattern of crystalline Ibrutinib Form D11 as obtained in example 19.

FIG. 15 illustrates a characteristic PXRD pattern of crystalline Ibrutinib Form D12 as obtained in example 20.

FIG. 16 illustrates a characteristic PXRD pattern of crystalline Ibrutinib Form D13 as obtained in example 21.

FIG. 17 illustrates a characteristic PXRD pattern of crystalline Ibrutinib Form D1a.

FIG. 18 illustrates a characteristic PXRD pattern of crystalline Ibrutinib Form D1 as obtained in example 22.

### DETAILED DESCRIPTION

In an embodiment, the present invention provides crystalline Ibrutinib, designated as Form D1, characterized by an x-ray powder diffraction pattern having peaks at about 10.11, 11.45, 18.47 and 20.89±0.20 degrees 2-theta, and also having peaks at about 5.04, 10.79, 23.10 and 26.60±0.20 degrees 2-theta.

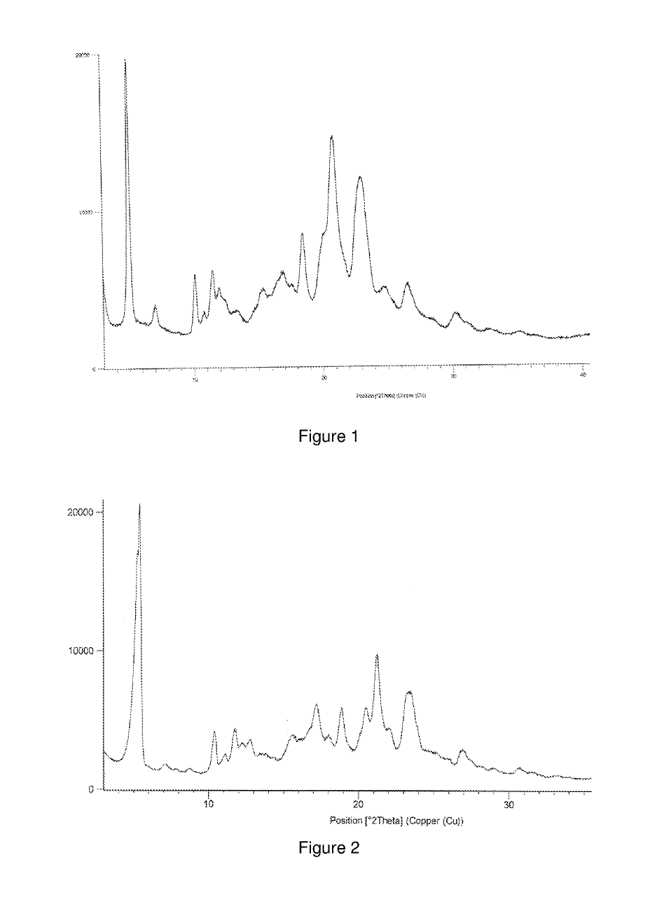

In an embodiment, the present invention provides crystalline Ibrutinib, designated as Form D1, characterized by an x-ray powder diffraction pattern having peaks located substantially as illustrated in the pattern of FIG. 1 or FIG. 2 or FIG. 18.

In another embodiment, the present invention encompasses a process for preparing crystalline Form D1 of Ibrutinib, comprising the steps of:

a) suspending ibrutinib in an alcohol at 10° C. to −40° C.;

b) maintaining the suspension at 10° C. to −40° C.; and

c) isolating at −10° C. to 10° C.

Suspending ibrutinib in step a) involves adding the alcohol solvent at 10° C. to −40° C. Ibrutinib used in step a) may be obtained by process known in the art. In a preferred embodiment, amorphous ibrutinib is used.

The alcohol that may be used in step a) is selected from a C₁₋₁₀ alcohol; preferably a C₁₋₅ alcohol such as methanol, ethanol, propanol, isopropanol, butanol, iso-butanol, pentanol or the like. In a preferred embodiment, the alcohol used is ethanol.

Step b) involves maintaining the suspension at a temperature of 10° C. to −40° C. The suspension is maintained for sufficient time to ensure the formation of crystalline ibrutinib Form D1.

Step c) involves isolating the crystalline ibrutinib Form D1. The crystalline ibrutinib Form D1 is isolated by techniques such as filtration, distillation, centrifugation, or slow evaporation or the like. In a preferred embodiment, crystalline ibrutinib Form D1 may be isolated by evaporation under vacuum and at a temperature of about −10° C. to 10° C. In a more preferred embodiment, the evaporation is carried out at −2° C. to 10° C.

In another embodiment, the present invention encompasses a process for preparing crystalline ibrutinib Form D1, comprising the steps of:

a) obtaining a solution of ibrutinib in an alcohol at 20° C. to 30° C.;

b) adding anti-solvent to the above solution at 2° C. to 10° C.;

c) maintaining at 2° C. to 10° C.; and

d) isolating at 2° C. to 30° C.

Obtaining a solution of ibrutinib in step a) involves adding alcohol to ibrutinib at 20° C. to 30° C. The alcohol that may be used in step a) is selected from C₁₋₁₀ alcohol; preferably a C₁₋₅ alcohol such as methanol, ethanol, propanol, isopropanol, butanol, iso-butanol, pentanol or the like. In a preferred embodiment, the alcohol used is methanol. Optionally, the solution of Ibrutinib is heated to 40-60° C. and filtered to make a particle free solution. Ibrutinib used in step a) may be obtained by process known in the art. Step b) involves adding anti-solvent to the ibrutinib solution at 2° C. to 10° C. Optionally, the anti-solvent is pre-cooled to 2 to 10° C. before adding to the Ibrutinib solution. In a preferred embodiment, the anti-solvent used is water.

Step d) involves isolating the crystalline Form D1a of Ibrutinib. The crystalline Form D1a may be isolated by techniques such as filtration, distillation, centrifugation, or slow evaporation or the like. In a preferred embodiment, crystalline Form D1a is isolated by filtration under vacuum and at a temperature of about 0° C. to 30° C.

In a specific embodiment, the crystalline Form D1a is dried to obtain crystalline form D1. Drying may be done using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In another embodiment, the present invention encompasses a process for preparing crystalline ibrutinib Form D1, comprising the steps of:

a) obtaining a solution of ibrutinib in an alcohol;

b) combining the solution of step a) with an anti-solvent; and

c) isolating crystalline form D1 of Ibrutinib.

In step a) any physical form of ibrutinib can be used, which may be crystalline or amorphous, for providing the solution of ibrutinib in alcohol. Obtaining a solution of ibrutinib in step a) involves adding alcohol to ibrutinib at about 0° C. to about 50° C. and more specifically at about 25° C. to about 35° C. The alcohol that may be used in step a) is selected from C₁₋₁₀ alcohol; preferably a C₁₋₅ alcohol such as methanol, ethanol, propanol, isopropanol, butanol, iso-butanol, pentanol or the like. In a preferred embodiment, the alcohol used is methanol. Optionally, the solution of Ibrutinib in alcohol is heated to 40-60° C. and filtered to make a particle free solution. Step b) involves combining the solution of step a) with an anti-solvent. In an embodiment, step b) may involve adding anti-solvent to the ibrutinib solution or conversely, adding ibrutinib solution to the anti-solvent at a temperature of about 0° C. to 15° C. and more specifically at about 2° C. to 10° C. Optionally, the anti-solvent is pre-cooled to 2° C. to 10° C. before mixing with the Ibrutinib solution. In a preferred embodiment, the anti-solvent used is water. In an embodiment of step B), the seed crystals of Ibrutinib Form D1 are optionally added to the mixture of anti-solvent and ibrutinib solution. The seed crystals can also be added either to the anti-solvent solution or to the ibrutinib solution. When the seed crystals are added, they are added in a quantity from about 0.5% w/w to about 15% w/w over the weight of ibrutinib. Specifically, the seed crystals are added in a quantity from about 1% to about 12% w/w and more specifically the seed crystals are added in a quantity from about 2% to about 10% w/w.

After combining the ibrutinib solution and anti-solvent, the mixture is stirred for about 30 minutes to about 24 hours or longer at the same temperature or at a temperature of about −5° C. to about 10° C.

In step c) the isolation of ibrutinib crystalline form D1 may optionally proceed through the intermediacy of crystalline Form D1a of Ibrutinib. Isolating crystalline form D1a of Ibrutinib may optionally involve one or more methods known in the art including removal of solvent by techniques known in the art e.g. evaporation, distillation, filtration of isolated solid and the like. Suitable temperatures for isolation may be less than about 25° C., less than about 10° C., less than about 0° C. or any other suitable temperatures. Filtration can be achieved by any means known in the art. Specifically, filtration can be achieved by using Buchner funnel or pressure nutsche filter (PNF) or jacketed agitated nutsche filter dryer (ANFD). While using jacketed ANFD equipment, the temperature of jacket may be maintained at about −20° C. to about 5° C. by circulating brine solution. After the filtration, the wet solid is optionally washed with cold water and suck-dried for about 30 minutes to about 24 hours or longer. In case of filtration by PNF, the suck-drying is achieved by applying a positive pressure of dry air or nitrogen. In case of filtration by jacketed ANFD, the suck-drying is achieved by applying vacuum while maintaining the atmospheric pressure by supplying dry air or nitrogen. In case of filtration by Buchner funnel, the suck-drying is achieved by applying a vacuum.

The solid obtained may be collected using techniques such as by scraping, or by shaking the container, or other techniques specific to the equipment used. The collected material is dried and drying may be suitably carried out using any of an air tray dryer, vacuum tray dryer, fluidized bed dryer, spin flash dryer, flash dryer, and the like. The drying may be carried out at atmospheric pressure or above, or under reduced pressures, specifically at temperatures less than about 80° C. and more specifically less than about 60° C. and most specifically less than about 40° C. The drying may be carried out for any time period required for obtaining a desired product quality, such as from about 5 minutes to about 24 hours, or longer.

The obtained Ibrutinib crystalline form D1 may optionally be subjected to a particle size reduction procedure to produce desired particle sizes and distributions by using techniques known in the art.

The particle size analysis may be done by any suitable instrument or techniques known in the art.

In an embodiment, the present invention provides crystalline Ibrutinib, designated as Form D2, characterized by an x-ray powder diffraction pattern having peaks at about 11.44, 12.51 and 26.64±0.20 degrees 2-theta, and also having peaks at about 5.00, 10.20, 20.87 and 23.15±0.20 degrees 2-theta.

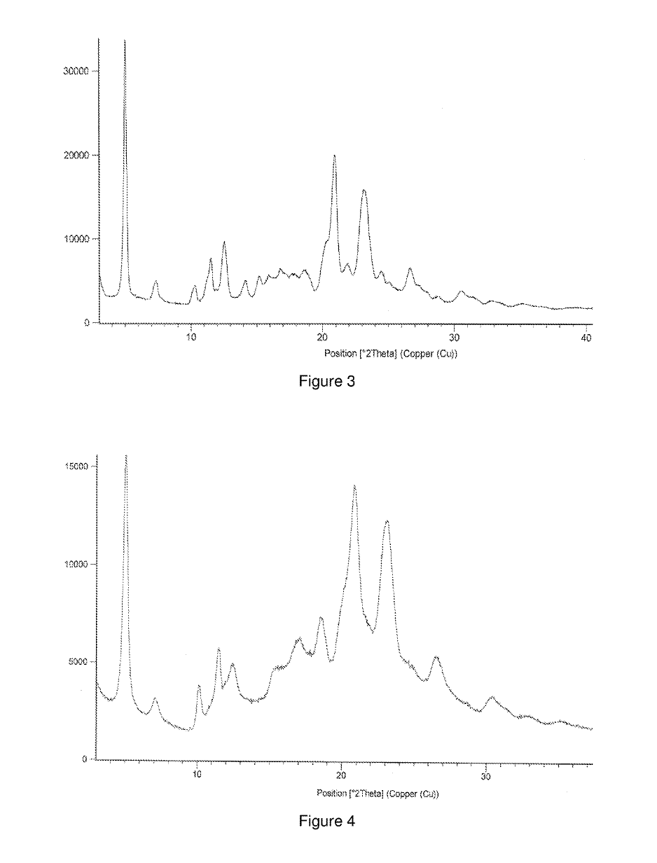

In an embodiment, the present invention provides crystalline Ibrutinib, designated as Form D2, characterized by an x-ray powder diffraction pattern having peaks located substantially as illustrated in the pattern of FIG. 3 or FIG. 4.

In another embodiment, the present invention encompasses a process for preparing crystalline ibrutinib Form D2, comprising the steps of:

a) suspending ibrutinib in a mixture of an alcohol and water at 10° C. to −40° C.;

b) maintaining the suspension at 10° C. to −40° C.;

c) isolating at −15° C. to −5° C., and

d) drying at −5° C. to 5° C.

Suspending ibrutinib in step a) involves adding alcohol and water to ibrutinib at 20° C. to −40° C. The ratio of alcohol and water may vary from about 60:40 to 95:5 by volume. In a preferred embodiment, the ratio is 80:20 by volume. Ibrutinib used in step a) may be obtained by process known in the art. In a preferred embodiment, amorphous ibrutinib is used.

The alcohol that may be used in step a) is selected from C₁₋₁₀ alcohol; preferably a C₁₋₅ alcohol such as methanol, ethanol, propanol, isopropanol, butanol, iso-butanol, pentanol or the like. In a preferred embodiment, the alcohol used is ethanol.

Suspending ibrutinib in step a) involves combining ibrutinib with ethanol and water at a temperature of 10° C. to −40° C.

Step b) involves maintaining the suspension at a temperature of 10° C. to −40° C.

Step c) involves isolating the crystalline ibrutinib Form D2a. The crystalline ibrutinib Form D2a is isolated by techniques such as filtration, distillation, centrifugation, or slow evaporation or the like.

Step d) involves drying the crystalline Ibrutinib Form D2a at a temperature of about −5° C. to 5° C. to obtain Form D2.

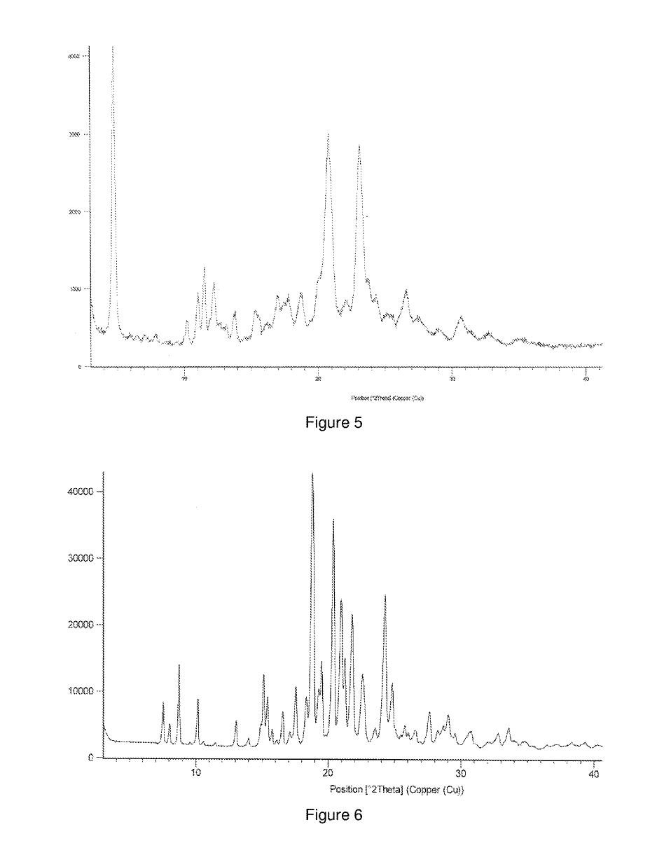

The crystalline Ibrutinib Form D2a characterized by PXRD pattern substantially as shown in FIG. 5.

In another embodiment, the present invention encompasses a process for preparing crystalline ibrutinib Form D1, comprising the steps of:

a) suspending ibrutinib in a mixture of an alcohol and water at 10° C. to −40° C.;

b) maintaining the suspension at 10° C. to −40° C.;

c) isolating at −10° C. to 10° C.; and

d) drying at 25° C. to 60° C.

Suspending ibrutinib in step a) involves adding alcohol and water to ibrutinib at 10° C. to −40° C. The ratio of alcohol and water may vary from about 60:40 to 95:5 by volume. In a preferred embodiment, the ratio is 80:20 by volume. Ibrutinib used in step a) may be obtained by process known in the art. In a preferred embodiment, amorphous ibrutinib is used.

The alcohol that may be used in step a) is selected from C₁₋₁₀ alcohol; preferably a C₁₋₅ alcohol such as methanol, ethanol, propanol, isopropanol, butanol, iso-butanol, pentanol or the like. In a preferred embodiment, the alcohol used is ethanol.

Suspending ibrutinib in step a) involves combining ibrutinib with ethanol and water at a temperature of 10° C. to −40° C.

Step b) involves maintaining the suspension at a temperature of 10° C. to −40° C.

Step c) involves isolating the crystalline Form D2 of Ibrutinib. The crystalline Form D2 may be isolated by techniques such as filtration, distillation, centrifugation, or slow evaporation or the like. In a preferred embodiment, compound is isolated by evaporation under vacuum and at a temperature of about −10° C. to 10° C.

Step d) involves drying the crystalline ibrutinib Form D2 at 25° C. to 60° C. to obtain crystalline ibrutinib Form D1.

Drying in step d) may be suitably carried out using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In an embodiment, the present invention provides crystalline Ibrutinib, designated as Form D3, characterized by an x-ray powder diffraction pattern having peaks at about 8.05, 8.77, 15.44, 21.80, 24.84, 27.65 and 29.10±0.20 degrees 2-theta, and also having peaks at about 7.56, 13.07, 15.15, 16.59, 18.89 and 21.27±0.20 degrees 2-theta. Ibrutinib Form D3 may be further characterized by x-ray powder diffraction pattern having additional peaks at about 10.17, 17.58, 18.36, 19.53, 20.42, 21.00, 22.54 and 24.31±0.20 degrees 2-theta.

Ibrutinib Form D3 characterized by x-ray powder diffraction pattern substantially as depicted in FIG. 6.

In an embodiment, ibrutinib Form D3 is acetophenone solvate.

In another embodiment, the present invention encompasses a process for preparing crystalline ibrutinib Form D3, comprising the steps of:

a) suspending ibrutinib in acetophenone at about 0° C. to −25° C.;

b) maintaining the suspension at about 0° C. to −25° C.; and

c) isolating at 25-30° C.

Suspending ibrutinib in step a) involves combining ibrutinib with acetophenone at a temperature of about 0° C. to −25° C. In a preferred embodiment, the temperature is about −10° C. Ibrutinib used in step a) may be obtained by process known in the art. In a preferred embodiment, amorphous ibrutinib is used.

Step b) involves maintaining the suspension at a temperature of about 0° C. to −25° C. The suspension is maintained for sufficient time to ensure the formation of crystalline Ibrutinib Form D3.

Step c) involves isolation of crystalline ibrutinib Form D3. The crystalline ibrutinib Form D3 is isolated by techniques such as filtration, distillation, centrifugation, or slow evaporation or the like. In a preferred embodiment, crystalline ibrutinib Form D3 may be isolated by filtration under vacuum at a temperature of about 20° C. to 30° C. In a more preferred embodiment, the filtration is at 25° C.

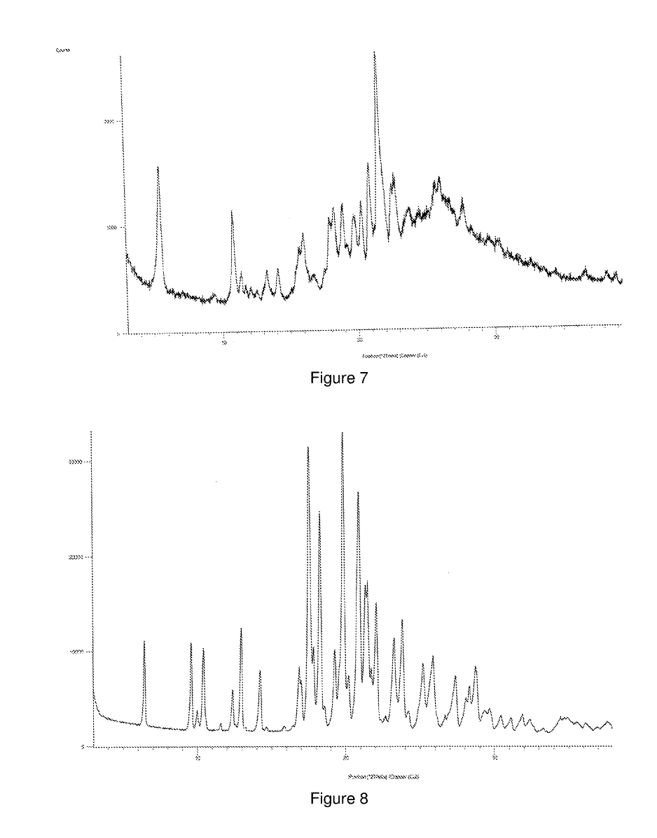

In an embodiment, the present invention provides crystalline Ibrutinib, designated as Form D4, characterized by an x-ray powder diffraction pattern having peaks at about 5.55, 10.87, 11.44, 13.31, 14.14 and 19.74±0.20 degrees 2-theta, and also having peaks at about 16.04, 18.36, 18.91, 20.35, 20.95 and 21.62±0.20 degrees 2-theta. Ibrutinib Form D4 characterized by x-ray powder diffraction pattern substantially as depicted in FIG. 7.

In an embodiment, ibrutinib form D4 is formamide solvate.

In another embodiment, the present invention encompasses a process for preparing crystalline ibrutinib Form D4, comprising the steps of:

a) suspending ibrutinib in formamide at about 10° C. to about −10° C.;

b) maintaining the suspension at about 10° C. to about −10° C.; and

c) isolating at 0° C. to −5° C.

Suspending ibrutinib in step a) involves combining ibrutinib with formamide at a temperature of about 10° C. to about −10° C. In a more preferred embodiment the temperature is about 0° C. to −10° C. Ibrutinib used in step a) may be obtained by process known in the art. In a preferred embodiment, amorphous ibrutinib is used.

Step b) involves maintaining the suspension at a temperature of about 0° C. to about −10° C. The suspension is maintained for sufficient time to ensure the formation of crystalline ibrutinib Form D4.

Step c) involves isolating the crystalline ibrutinib Form D4. The crystalline ibrutinib Form D4 is isolated by techniques such as filtration, distillation, centrifugation, or slow evaporation or the like. In a preferred embodiment, crystalline ibrutinib Form D4 may be isolated by evaporation of solvent followed by nitrogen purging at a temperature of about 0° C. to −5° C. In a more preferred embodiment, the evaporation is at −5° C.

In an embodiment, the present invention provides crystalline Ibrutinib, designated as Form D5, characterized by an x-ray powder diffraction pattern having peaks at about 6.50, 9.63, 10.45, 12.41, 13.02, 14.28, 19.94, 23.88, 25.93, 27.44 and 28.85±0.20 degrees 2-theta, and also having peaks at about 17.64, 18.37, 21.00, 23.31 and 25.23±0.20 degrees 2-theta. Ibrutinib Form D5 may be further characterized by x-ray powder diffraction pattern having additional peaks at about 10.01 and 19.33±0.20 degrees 2-theta. Ibrutinib Form D5 characterized by x-ray powder diffraction pattern substantially as depicted in FIG. 8.

In an embodiment, Ibrutinib form D5 is acetone solvate.

In another embodiment, the present invention encompasses a process for preparing crystalline ibrutinib Form D5, comprising the steps of:

a) suspending ibrutinib in acetone at about 20° C. to 30° C.;

b) maintaining the suspension at about 20° C. to 30° C.; and

c) isolating at 20° C. to 30° C.

Suspending ibrutinib in step a) involves combining ibrutinib with acetone at a temperature of 20° C. to 30° C. In a preferred embodiment, the temperature is 25° C. Ibrutinib used in step a) may be obtained by process known in the art. In a preferred embodiment, form D3 of ibrutinib is used.

Step b) involves maintaining the suspension at a temperature of about 20° C. to 30° C. The suspension is maintained for sufficient time to ensure the formation of crystalline ibrutinib Form D5.

Step c) involves isolating the crystalline ibrutinib Form D5. The crystalline ibrutinib Form D5 is isolated by techniques such as filtration, distillation, centrifugation, or slow evaporation or the like. In a preferred embodiment, crystalline ibrutinib Form D5 may be isolated by evaporation followed by nitrogen purging at a temperature of about 20 to 25° C. In a more preferred embodiment, the evaporation is at 25° C.

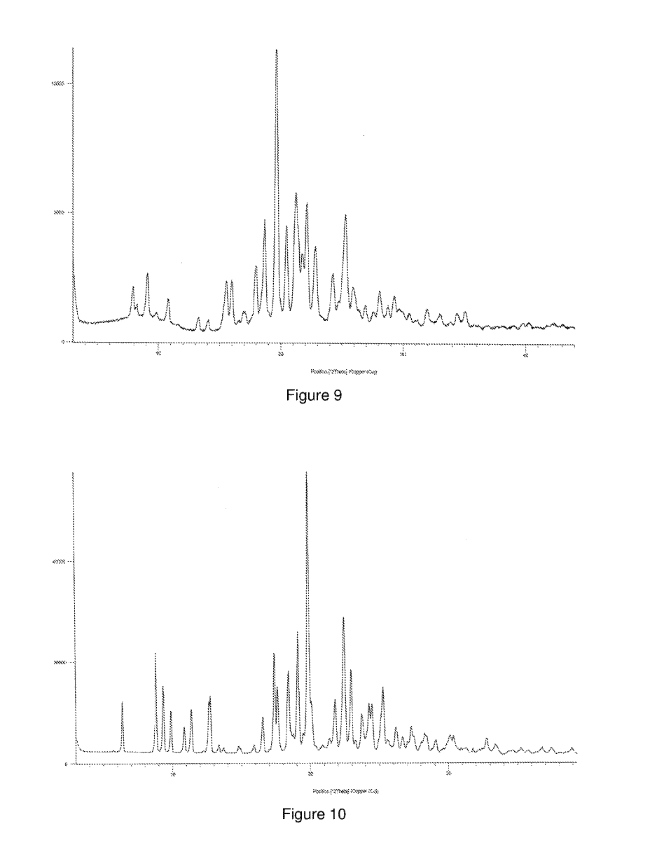

In an embodiment, the present invention provides crystalline Ibrutinib, designated as Form D6, characterized by an x-ray powder diffraction pattern having peaks at about 7.97, 9.16, 10.88, 16.06, 18.76, 19.72, 22.17, 22.77, 26.96 and 28.08±0.20 degrees 2-theta, and also having peaks at about 13.31, 14.08, 15.61, 17.90, 21.27 and 25.38±0.20 degrees 2-theta. Ibrutinib Form D6 may be further characterized by x-ray powder diffraction pattern having additional peaks at about 20.55, 24.32 and 25.87±0.20 degrees 2-theta. Ibrutinib Form D6 characterized by x-ray powder diffraction pattern substantially as depicted in FIG. 9.

In an embodiment, Ibrutinib form D6 is chlorobenzene solvate.

In another embodiment, the present invention encompasses a process for preparing crystalline ibrutinib Form D6, comprising the steps of:

a) suspending ibrutinib in chlorobenzene at about 20° C. to about −40° C.;

b) maintaining the suspension at about 20° C. to about −40° C.; and

c) isolating at about −10° C. to about 20° C.

Suspending ibrutinib in step a) involves combining ibrutinib with chlorobenzene at a temperature of about 20° C. to about −25° C. Ibrutinib used in step a) may be obtained by process known in the art. In a preferred embodiment, Form C of ibrutinib disclosed in WO2013184572 is used.

Step b) involves maintaining the suspension at a temperature of 20° C. to −25° C. The suspension is maintained for sufficient time to ensure the formation of crystalline ibrutinib Form D6.

Step c) involves isolating the crystalline ibrutinib Form D6. The crystalline ibrutinib Form D6 is isolated by techniques such as filtration, distillation, centrifugation, or slow evaporation or the like. In a preferred embodiment, crystalline ibrutinib Form D6 may be isolated by evaporation followed by nitrogen purging at a temperature of about −10° C. to 20° C. In a more preferred embodiment, the evaporation is at 0° C.

In an embodiment, the present invention provides crystalline Ibrutinib, designated as Form D7, characterized by an x-ray powder diffraction pattern having peaks at about 8.83, 9.37, 9.92, 10.87, 11.40, 18.48, 19.92, 21.83, 23.71 and 25.29±0.20 degrees 2-theta, and also having peaks at about 6.40, 16.60, 17.43, 17.67, 19.17, and 24.26±0.20 degrees 2-theta. Ibrutinib Form D7 may be further characterized by x-ray powder diffraction having additional peaks at about 13.37, 13.73, 15.93, 22.51 and 22.99±0.20 degrees 2-theta. Ibrutinib Form D7 characterized by x-ray powder diffraction pattern substantially as depicted in FIG. 10.

In an embodiment, Ibrutinib form D7 is dimethylacetamide solvate.

In another embodiment, the present invention encompasses a process for preparing crystalline ibrutinib Form D7, comprising the steps of:

a) suspending ibrutinib in dimethylacetamide at about 20° C. to about −40° C.;

b) maintaining the suspension at about 20° C. to about −40° C.; and

c) isolating at about −10° C. to 20° C.

Suspending ibrutinib in step a) involves combining ibrutinib with dimethylacetamide at a temperature of about 20° C. to about −40° C. Ibrutinib used in step a) may be obtained by process known in the art. In a preferred embodiment, Form C of ibrutinib disclosed in WO2013184572 is used.

Step b) involves maintaining the suspension at a temperature of about 20° C. to about −40° C. The suspension is maintained for sufficient time to ensure the formation of crystalline ibrutinib Form D7.

Step c) involves isolating the crystalline ibrutinib Form D7. The crystalline ibrutinib Form D7 is isolated by techniques such as filtration, distillation, centrifugation, or slow evaporation or the like. In a preferred embodiment, crystalline ibrutinib Form D7 may be isolated by evaporation followed by nitrogen purging at a temperature of about −10° C. to 20° C. In a more preferred embodiment, the evaporation is at 0-5° C.

In an embodiment, the present invention provides crystalline Ibrutinib, designated as Form D8, characterized by an x-ray powder diffraction pattern having peaks at about 9.27, 9.69, 10.97, 14.24, 24.83, 25.83, 28.21 and 28.79±0.20 degrees 2-theta, and also having peaks at about 5.03, 7.13, 13.23, 16.00, 17.26, 17.59 21.60 and 22.75±0.20 degrees 2-theta. Ibrutinib Form D8 may be further characterized by x-ray powder diffraction having additional peaks at about 10.17, 18.51, 19.56, 20.47 and 22.07±0.20 degrees 2-theta. Ibrutinib Form D8 characterized by x-ray powder diffraction pattern substantially as depicted in FIG. 11.

In an embodiment, Ibrutinib form D8 is acetone solvate.

In another embodiment, the present invention encompasses a process for preparing crystalline ibrutinib Form D8, comprising the steps of:

a) suspending ibrutinib in acetone at about 20° C. to about −40° C.;

b) maintaining at about 20° C. to about −40° C.; and

c) isolating at −10° C. to 10° C.

Suspending ibrutinib in step a) involves combining ibrutinib with acetone at a temperature of about 20° C. to about −40° C. Ibrutinib used in step a) may be obtained by process known in the art. In a preferred embodiment, Form C of ibrutinib disclosed in WO2013184572 is used.

Step b) involves maintaining the suspension at a temperature of about 20° C. to about −40° C. The suspension is maintained for sufficient time to ensure the formation of crystalline ibrutinib Form D8.

Step c) involves isolating the crystalline ibrutinib Form D8. The crystalline ibrutinib Form D8 is isolated by techniques such as filtration, distillation, centrifugation, or slow evaporation or the like. In a preferred embodiment, crystalline ibrutinib Form D8 may be isolated by evaporation followed by nitrogen purging at a temperature of about −10° C. to 10° C. In a more preferred embodiment, the evaporation is at about 0-5° C.

In an embodiment, the present invention provides crystalline Ibrutinib, designated as Form D9, characterized by an x-ray powder diffraction pattern having peaks at about 7.71, 12.61, 13.14, 22.69 and 23.65±0.20 degrees 2θ; with peaks at about 8.04, 19.07, 20.20 and 31.79±0.20 degrees 2θ. Crystalline Ibrutinib Form D9 is further characterized by peaks at about 18.63, 20.81 and 21.51±0.20 degrees 2θ.

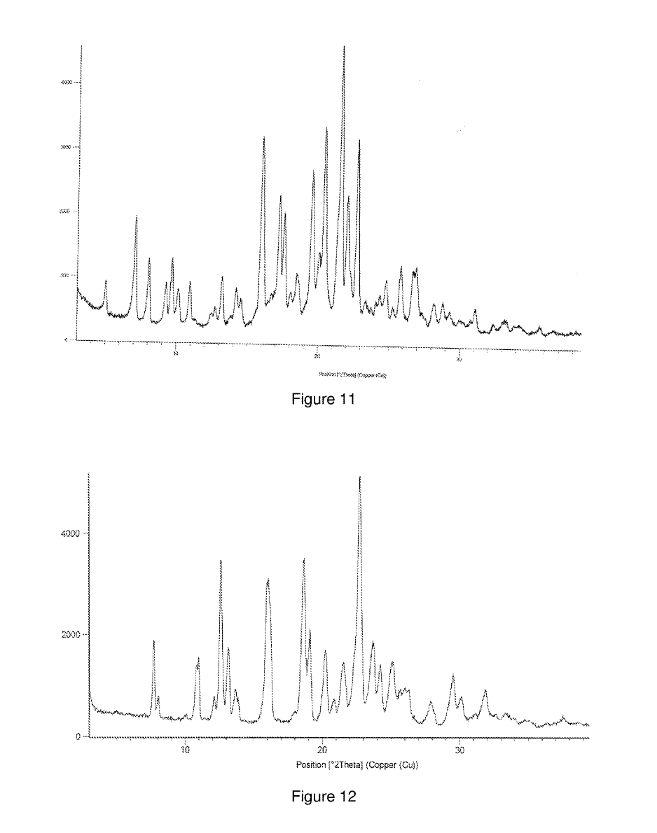

In an embodiment, the present invention provides crystalline Ibrutinib, designated as Form D9, characterized by an x-ray powder diffraction pattern having peaks located substantially as illustrated in the pattern of FIG. 12.

In another embodiment, the present invention encompasses a process for preparing crystalline Form D9 of Ibrutinib, comprising the steps of:

a) suspending ibrutinib in a solvent or mixture of solvents at 20° C. to 70° C.;

b) maintaining the suspension at 20° C. to 70° C.; and

c) isolating at 20° C. to 70° C.

Obtaining the suspension of ibrutinib in step a) involves adding a solvent or mixture of solvents at 20° C. to 70° C. Ibrutinib used in step a) may be obtained by process known in the art. In a preferred embodiment, amorphous ibrutinib is used.

The solvent or mixture of solvents that may be used in step a) is selected from a C₁₋₁₀ alcohol; preferably a C₁₋₅ alcohol such as methanol, ethanol, propanol, isopropanol, butanol, iso-butanol, pentanol or the like; ethers such as diethyl ether, diisopropyl ether, t-butyl methyl ether, di-butyl ether, tetrahydrofuran, 1, 2-dimethoxyethane, 2-methoxyethanol, 2-ethoxyethanol, anisole, 1, 4-dioxane or the like; esters, such as ethyl formate, methyl acetate, ethyl acetate, propyl acetate, butyl acetate, methyl propanoate, ethyl propanoate, methyl butanoate, ethyl butanoate, dimethyl carbonate or the like; aliphatic or alicyclic hydrocarbons such as hexane, heptane, pentane, cyclohexane, methyl cyclohexane or the like; aromatic hydrocarbons such as toluene, xylene or the like; or any mixtures thereof. In a preferred embodiment the solvent is methanol. In another preferred embodiment, the mixture of solvents is butanol and heptane.

Step b) involves maintaining the suspension at a temperature of 20° C. to 70° C. The suspension is maintained for sufficient time to ensure the formation of crystalline ibrutinib Form D9.

Step c) involves isolating the crystalline ibrutinib Form D9. The crystalline ibrutinib Form D9 is isolated by techniques such as filtration, distillation, centrifugation, or slow evaporation or the like. In a preferred embodiment, crystalline ibrutinib Form D9 may be isolated by filtration under vacuum and at a temperature of about 20° C. to 70° C.

Crystalline Form D9 obtained in step c) may be optionally dried using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In an embodiment, the present invention provides crystalline Ibrutinib, designated as Form D10, characterized by an x-ray powder diffraction pattern having peaks at about 6.62, 10.57, 13.30, 17.17, 19.97, 21.27, 25.07 and 29.59±0.20 degrees 2θ; with peaks at about 10.19, 15.32, 18.09, 18.80, 21.95, 26.35 and 26.85±0.20 degrees 2θ. Crystalline Ibrutinib Form D10 is further characterized by peaks at about 9.68, 19.28 and 28.08±0.20 degrees 2θ.

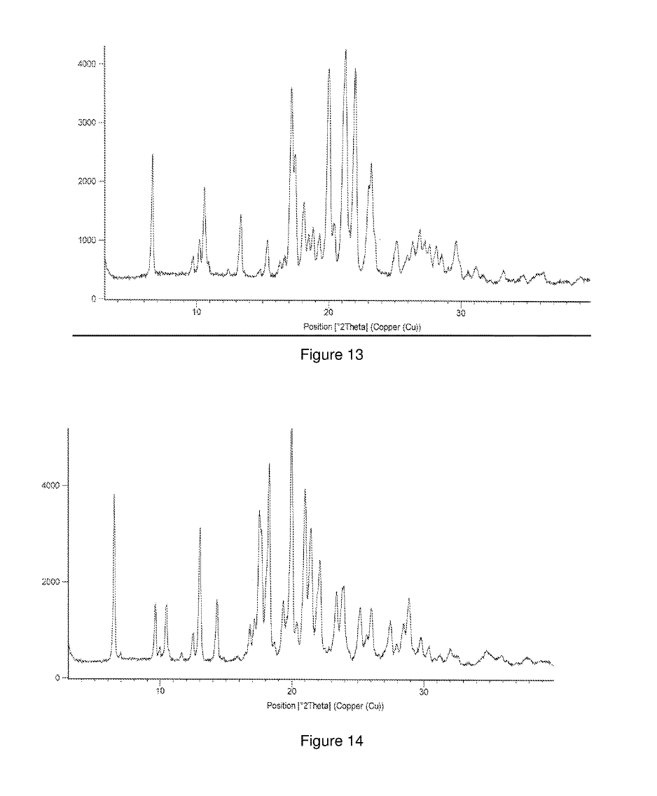

In an embodiment, the present invention provides crystalline Ibrutinib, designated as Form D10, characterized by an x-ray powder diffraction pattern having peaks located substantially as illustrated in the pattern of FIG. 13.

In an embodiment, Ibrutinib form D10 is a 1, 2-dimethoxyethane solvate.

In another embodiment, the present invention encompasses a process for preparing crystalline ibrutinib Form D10, comprising the steps of:

a) suspending ibrutinib in a solvent or mixture of solvents at 2° C. to 25° C.;

b) maintaining the suspension at 2° C. to 25° C.;

c) isolating at 2° C. to 25° C.

Obtaining the suspension of ibrutinib in step a) involves adding a solvent or mixture of solvents at 2° C. to 25° C. Ibrutinib used in step a) may be obtained by process known in the art.

The solvent or mixture of solvents that may be used in step a) is selected from a C₁₋₁₀ alcohol; preferably a C₁₋₅ alcohol such as methanol, ethanol, propanol, isopropanol, butanol, iso-butanol, pentanol or the like; ethers such as diethyl ether, diisopropyl ether, t-butyl methyl ether, di-butyl ether, tetrahydrofuran, 1, 2-dimethoxyethane, 2-methoxyethanol, 2-ethoxyethanol, anisole, 1,4-dioxane or the like; esters, such as ethyl formate, methyl acetate, ethyl acetate, propyl acetate, butyl acetate, methyl propanoate, ethyl propanoate, methyl butanoate, ethyl butanoate, dimethyl carbonate or the like; aliphatic or alicyclic hydrocarbons such as hexane, heptane, pentane, cyclohexane, methyl cyclohexane or the like; aromatic hydrocarbons such as toluene, xylene or the like; or any mixtures thereof. In a preferred embodiment the solvent is 1, 2-dimethoxyethane.

Step b) involves maintaining the suspension at a temperature of 2° C. to 25° C. The suspension is maintained for sufficient time to ensure the formation of crystalline ibrutinib Form D10.

Step c) involves isolating the crystalline ibrutinib Form D10. The crystalline ibrutinib Form D10 is isolated by techniques such as filtration, distillation, centrifugation, or slow evaporation or the like. In a preferred embodiment, crystalline ibrutinib Form D10 may be isolated by filtration under vacuum and at a temperature of about 2° C. to 25° C.

Crystalline Form D10 obtained in step c) may be optionally dried using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In an embodiment, the present invention provides crystalline Ibrutinib, designated as Form D11, characterized by an x-ray powder diffraction pattern having peaks at about 6.49, 9.60, 10.44, 12.99, 14.28, 18.23, 19.94, 23.85, 25.99, 27.39 and 28.85±0.20 degrees 2θ; with peaks at about 12.46, 20.97, 23.34 and 25.13±0.20 degrees 2θ.

In an embodiment, the present invention provides crystalline Ibrutinib, designated as Form D11, characterized by an x-ray powder diffraction pattern having peaks located substantially as illustrated in the pattern of FIG. 14.

In an embodiment, Ibrutinib form D11 is an anisole solvate.

In another embodiment, the present invention encompasses a process for preparing crystalline ibrutinib Form D11, comprising the steps of:

a) suspending ibrutinib in a solvent or mixture of solvents at 2° C. to 25° C.;

b) maintaining the suspension at 2° C. to 25° C.;

c) isolating at 2° C. to 25° C.

Obtaining the suspension of ibrutinib in step a) involves adding a solvent or mixture of solvents at 2° C. to 25° C. Ibrutinib used in step a) may be obtained by process known in the art.

The solvent or mixture of solvents that may be used in step a) is selected from a C₁₋₁₀ alcohol; preferably a C₁₋₅ alcohol such as methanol, ethanol, propanol, isopropanol, butanol, iso-butanol, pentanol or the like; ethers such as diethyl ether, diisopropyl ether, t-butyl methyl ether, di-butyl ether, tetrahydrofuran, 1, 2-dimethoxyethane, 2-methoxyethanol, 2-ethoxyethanol, anisole, 1,4-dioxane or the like; esters, such as ethyl formate, methyl acetate, ethyl acetate, propyl acetate, butyl acetate, methyl propanoate, ethyl propanoate, methyl butanoate, ethyl butanoate, dimethyl carbonate or the like; aliphatic or alicyclic hydrocarbons such as hexane, heptane, pentane, cyclohexane, methyl cyclohexane or the like; aromatic hydrocarbons such as toluene, xylene or the like; or any mixtures thereof. In a preferred embodiment the solvent is anisole.

Step b) involves maintaining the suspension at a temperature of 2° C. to 20° C. The suspension is maintained for sufficient time to ensure the formation of crystalline ibrutinib Form D11.

Step c) involves isolating the crystalline ibrutinib Form D11. The crystalline ibrutinib Form D11 is isolated by techniques such as filtration, distillation, centrifugation, or slow evaporation or the like. In a preferred embodiment, crystalline ibrutinib Form D11 may be isolated by filtration under vacuum and at a temperature of about 2° C. to 25° C.

Crystalline Form D11 obtained in step c) may be optionally dried using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In an embodiment, the present invention provides crystalline Ibrutinib, designated as Form D12, characterized by an x-ray powder diffraction pattern having peaks at about 9.79, 12.95, 14.08, 19.84, 21.82, 22.48, 23.71, 25.55 and 26.92±0.20 degrees 2θ; with peaks at about 6.46, 10.24, 15.97, 17.65 and 24.71±0.20 degrees 2θ. Crystalline Ibrutinib Form D12 is further characterized by peaks at about 18.17 and 20.60±0.20 degrees 2θ.

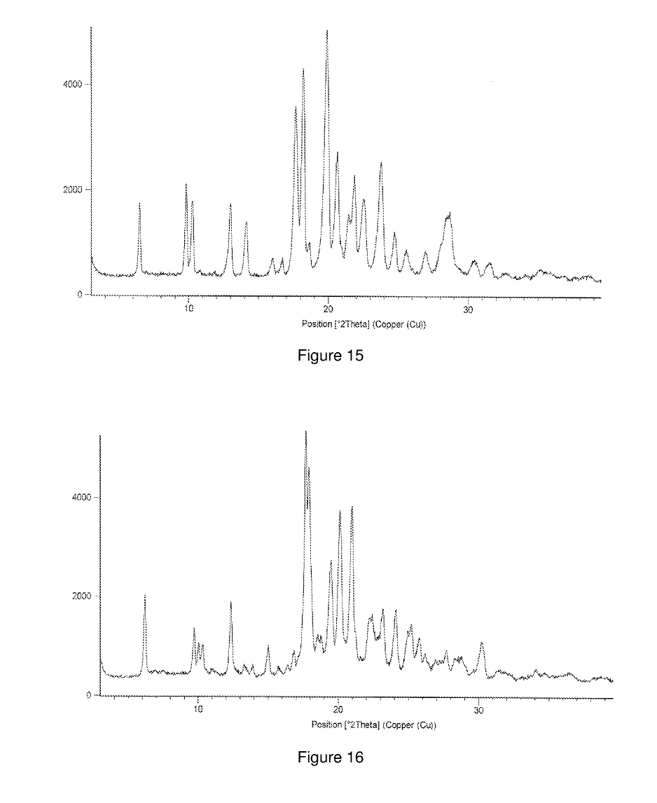

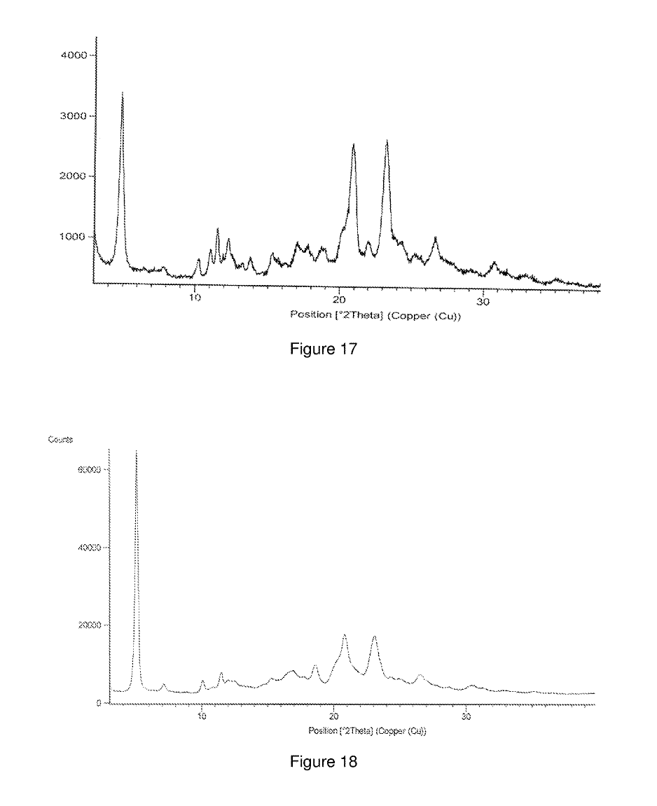

In an embodiment, the present invention provides crystalline Ibrutinib, designated as Form D12, characterized by an x-ray powder diffraction pattern having peaks located substantially as illustrated in the pattern of FIG. 15.

In an embodiment, Ibrutinib form D12 is a dimethyl carbonate solvate.

In another embodiment, the present invention encompasses a process for preparing crystalline ibrutinib Form D12, comprising the steps of:

a) suspending ibrutinib in a solvent or mixture of solvents at 2° C. to 25° C.;

b) maintaining the suspension at 2° C. to 25° C.;

c) isolating at 2° C. to 25° C.

Obtaining the suspension of ibrutinib in step a) involves adding a solvent or mixture of solvents at 2° C. to 25° C. Ibrutinib used in step a) may be obtained by process known in the art.

The solvent or mixture of solvents that may be used in step a) is selected from a C₁₋₁₀ alcohol; preferably a C₁₋₅ alcohol such as methanol, ethanol, propanol, isopropanol, butanol, iso-butanol, pentanol or the like; ethers such as diethyl ether, diisopropyl ether, t-butyl methyl ether, di-butyl ether, tetrahydrofuran, 1, 2-dimethoxyethane, 2-methoxyethanol, 2-ethoxyethanol, anisole, 1,4-dioxane or the like; esters, such as ethyl formate, methyl acetate, ethyl acetate, propyl acetate, butyl acetate, methyl propanoate, ethyl propanoate, methyl butanoate, ethyl butanoate, dimethyl carbonate or the like; aliphatic or alicyclic hydrocarbons such as hexane, heptane, pentane, cyclohexane, methyl cyclohexane or the like; aromatic hydrocarbons such as toluene, xylene or the like; or any mixtures thereof. In a preferred embodiment, the solvent is dimethyl carbonate.

Step b) involves maintaining the suspension at a temperature of 2° C. to 25° C. The suspension is maintained for sufficient time to ensure the formation of crystalline ibrutinib Form D12.

Step c) involves isolating the crystalline ibrutinib Form D12. The crystalline ibrutinib Form D12 is isolated by techniques such as filtration, distillation, centrifugation, or slow evaporation or the like. In a preferred embodiment, crystalline ibrutinib Form D12 may be isolated by filtration under vacuum and at a temperature of about 2° C. to 25° C.

Crystalline Form D12 obtained in step c) may be optionally dried using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In an embodiment, the present invention provides crystalline Ibrutinib, designated as Form D13, characterized by an x-ray powder diffraction pattern having peaks at about 9.69, 10.30, 12.32, 17.66 and 24.05±0.20 degrees 2θ; with peaks at about 6.18, 10.01, 14.97 and 23.13±0.20 degrees 2θ. Crystalline Ibrutinib Form D13 is further characterized by peaks at about 16.78, 17.92 and 19.42±0.20 degrees 2θ.

In an embodiment, the present invention provides crystalline Ibrutinib, designated as Form D13, characterized by an x-ray powder diffraction pattern having peaks located substantially as illustrated in the pattern of FIG. 16.

In an embodiment, Ibrutinib form D13 is a 1, 4-dioxane solvate.

In another embodiment, the present invention encompasses a process for preparing crystalline ibrutinib Form D13, comprising the steps of:

a) suspending ibrutinib in a solvent or mixture of solvents at 2° C. to 25° C.;

b) maintaining the suspension at 2° C. to 25° C.;

c) isolating at 2° C. to 25° C.

Obtaining the suspension of ibrutinib in step a) involves adding a solvent or mixture of solvents at 2° C. to 25° C. Ibrutinib used in step a) may be obtained by process known in the art.

The solvent or mixture of solvents that may be used in step a) is selected from a C₁₋₁₀ alcohol; preferably a C₁₋₅ alcohol such as methanol, ethanol, propanol, isopropanol, butanol, iso-butanol, pentanol or the like; ethers such as diethyl ether, diisopropyl ether, t-butyl methyl ether, di-butyl ether, tetrahydrofuran, 1, 2-dimethoxyethane, 2-methoxyethanol, 2-ethoxyethanol, anisole, 1,4-dioxane or the like; esters, such as ethyl formate, methyl acetate, ethyl acetate, propyl acetate, butyl acetate, methyl propanoate, ethyl propanoate, methyl butanoate, ethyl butanoate, dimethyl carbonate or the like; aliphatic or alicyclic hydrocarbons such as hexane, heptane, pentane, cyclohexane, methyl cyclohexane or the like; aromatic hydrocarbons such as toluene, xylene or the like; or any mixtures thereof. In a preferred embodiment, the solvent is 1,4-dioxane.

Step b) involves maintaining the suspension at a temperature of 2° C. to 25° C. The suspension is maintained for sufficient time to ensure the formation of crystalline ibrutinib Form D13.

Step c) involves isolating the crystalline ibrutinib Form D13. The crystalline ibrutinib Form D13 is isolated by techniques such as filtration, distillation, centrifugation, or slow evaporation or the like. In a preferred embodiment, crystalline ibrutinib Form D13 may be isolated by filtration under vacuum and at a temperature of about 2° C. to 25° C.

Crystalline Form D13 obtained in step c) may be optionally dried using any equipment such as a gravity oven, tray dryer, vacuum oven, Rotavapor®, air tray dryer, fluidized bed dryer, spin flash dryer, flash dryer, and the like. In an embodiment, the drying may be carried out at atmospheric pressure or under reduced pressure. In an embodiment, the drying may be carried out at a temperature of about 60° C., at a temperature of about 50° C., at a temperature of about 40° C. or at a temperature of about 30° C. The drying may be carried out for any time periods required for obtaining a desired quality, such as from about 15 minutes to several hours, or longer.

In one embodiment, the present invention provides pharmaceutical composition comprising crystalline form of ibrutinib designated as Form D1, Form D1a, Form D2, Form D2a, Form D3, Form D4, Form D5, Form D6, Form D7, Form D8, Form D9, Form D10, Form D11, Form D12 and Form D13 and at least one pharmaceutically acceptable excipient.

Pharmaceutically acceptable excipients include, but are not limited to, suitable surface modifiers. Such excipients include various polymers, low molecular weight oligomers, natural products, and surfactants.

The crystalline forms of ibrutinib designated as Form D1, Form D1a, Form D2, Form D2a, Form D3, Form D4, Form D5, Form D6, Form D7, Form D8, Form D9, Form D10, Form D11, Form D12 and Form D13 of the present invention have advantageous properties selected from at least one: chemical purity, stability—such as storage stability, stability to dehydrate, stability to polymorphic conversion, flowability, solubility, morphology or crystal habit, low hygroscopicity and low content of residual solvents.

Certain specific aspects and embodiments of the present invention will be explained in more detail with reference to the following examples, which are provided only for purposes of illustration and should not be construed as limiting the scope of the present application in any manner.

### General Description of the PXRD Equipment:

X-ray diffraction was measured using PANalytical X-ray diffractometer, Model: X′Pert PRO. System description: CuK-Alpha 1 wavelength=1.54060, voltage 45 kV, current 40 mA, divergence slit=0.5°; Sample stage=Reflection-Transmission Spinner. Scan type: Continuous; Detector—X′Celerator; Measurement parameters: Start Position [° 2Th.]: 3; End Position [° 2Th.]: 40; Step Size [° 2Th.]:0.0170; Scan Step Time [s]: 170.1800.

### EXAMPLES

### Example 1: Preparation of Crystalline Ibrutinib Form D1

1.0 g of ibrutinib was charged into the round bottom flask at 25-30° C. and cooled to −18° C. 3.1 mL of ethanol-water mixture (80:20) was charged to the above flask at −18° C. The contents were maintained for 18 hours at −15 to −20° C. The contents were distilled at 0° C. and dried in vacuum tray drier at 54° C. for 6-7 hours to obtain the title compound.

The crystal Form D1 is characterized by PXRD given in FIG. 1.

### Example 2: Preparation of Crystalline Ibrutinib Form D1

40 mg of ibrutinib was taken into the well of high throughput screening instrument at 25-30° C. 0.25 mL of ethanol was added to the above well at the same temperature. The contents were vortexed at −25° C. for 13 hours. The compound was dried under vacuum followed by nitrogen purging at −2° C. to obtain the title compound.

The crystal Form D1 is characterized by PXRD given in FIG. 2.

### Example 3: Preparation of Crystalline Ibrutinib Form D2

1.0 g of ibrutinib was charged into the round bottom flask at 25-30° C. and cooled to −18° C. 3.1 mL of ethanol-water mixture (80:20) was charged to the above flask at −18° C. The contents were maintained for 18 hours at −15 to −20° C. The contents were distilled at 0° C. and dried at 2° C. for 4-5 hours to obtain the title compound.

The crystal Form D2 is characterized by PXRD given in FIG. 3.

### Example 4: Preparation of Crystalline Ibrutinib Form D2

1.5 g of ibrutinib was charged into the round bottom flask at 25-30° C. and cooled to −18° C. 4.6 mL of ethanol-water mixture (80:20) was charged to the above flask at −18° C. The contents were maintained for 15 to 18 hours at −15 to −20° C. The contents were distilled at 0° C. under reduced pressure for 2-4 hours. The distilled compound was dried under reduced pressure at 52° C. for 4-6 hours to obtain the title compound.

The crystal Form D2 is characterized by PXRD given in FIG. 4.

### Example 5: Preparation of Crystalline Ibrutinib Form D2

80 mg of ibrutinib was taken into the well of high throughput screening instrument at 25-30° C. and cooled to −25° C. 0.25 mL of ethanol-water mixture (95:5) was added to the above well at −25° C. The contents were vortexed at −25° C. for 13 hours. The compound was dried under vacuum followed by nitrogen purging at 0° C. to obtain the title compound.

### Example 6: Preparation of Crystalline Ibrutinib Form D2

80 mg of ibrutinib was taken into the well of high throughput screening instrument at 25-30° C. and cooled to −25° C. 0.25 mL of ethanol-water mixture (90:10) was added to the above well −25° C. The contents were vortexed at −25° C. for 13 hours. The compound was dried under vacuum followed by nitrogen purging at 0° C. to obtain the title compound.

### Example 7: Preparation of Crystalline Ibrutinib Form D2

80 mg of ibrutinib was taken into the well of high throughput screening instrument at 25-30° C. and cooled to −25° C. 0.25 mL of ethanol-water mixture (85:15) was added to the above well at −25° C. The contents were vortexed at −25° C. for 13 hours. The compound was dried under vacuum followed by nitrogen purging at 0° C. to obtain the title compound.

### Example 8: Preparation of Crystalline Ibrutinib Form D3

2.5 g of ibrutinib was charged into the round bottom flask and cooled to −10° C. 9.0 mL of acetophenone was charged to the above flask at −10° C. The contents were cooled to 0 to −10° C. and maintained for 13-18 hours at −10° C. The contents were filtered at 25° C. to obtain the title compound.

The crystal Form D3 is characterized by PXRD given in FIG. 6.

### Example 9: Preparation of Crystalline Ibrutinib Form D4

80 mg of ibrutinib was taken into the well of high throughput screening instrument at 25-30° C. 0.3 mL of formamide was added to the above well at 0° C. The contents were vortexed at 0° C. for 18 hours. The compound was dried under vacuum followed by nitrogen purging at −5° C. to obtain the title compound.

The crystal Form D4 is characterized by PXRD given in FIG. 7.

### Example 10: Preparation of Crystalline Ibrutinib Form D5

0.25 g of ibrutinib was charged into the round bottom flask at 25° C. to 30° C. 0.6 mL of acetone was charged to the above flask. The contents were stirred at 25-30° C. and maintained for 1-2 hours. The solvent was evaporated using Nitrogen to obtain the title compound.

The crystal Form D5 is characterized by PXRD given in FIG. 8.

### Example 11: Preparation of Crystalline Ibrutinib Form D6

50 mg of ibrutinib was taken into the well of high throughput screening instrument at 25-30° C. and cooled to −25° C. 0.25 mL of chlorobenzene was added to the above well at −25° C. The contents were vortexed at −25° C. for 13 hours. The compound was dried under vacuum followed by nitrogen purging at 0° C. to obtain the title compound.

The crystal Form D6 is characterized by PXRD given in FIG. 9.

### Example 12: Preparation of Crystalline Ibrutinib Form D7

50 mg of ibrutinib was taken into the well of high throughput screening instrument at 25-30° C. and cooled to −25° C. 0.25 mL of dimethylacetamide was added to the above well at −25° C. The contents were vortexed at −25° C. for 13 hours. The compound was dried under vacuum followed by nitrogen purging at 0° C. to obtain the title compound.

The crystal Form D7 is characterized by PXRD given in FIG. 10.

### Example 13: Preparation of Crystalline Ibrutinib Form D8

50 mg of ibrutinib was taken into the well of high throughput screening instrument at 25-30° C. and cooled to −25° C. 0.25 mL of acetone was added to the above well at −25° C. The well was vortexed at −25° C. for 13 hours. The solvent was evaporated under reduced pressure at 0° C. to obtain the title compound.

The crystal Form D8 is characterized by PXRD given in FIG. 11.

### Example 14: Preparation of Crystalline Ibrutinib Form D1

5.0 g of ibrutinib was charged into the round bottom flask at 25-30° C. 200 mL of methanol was charged into the flask and the contents were heated to 50-60° C. The solution was made particle free and cooled to 5-10° C. 200 mL of demineralized water, pre-cooled to 2-10° C., was added slowly to the above flask. The contents of the flask were maintained for 1-3 hours at 5-15° C. and filtered using vacuum pump to obtain crystalline Form D1a. The crystalline Form D1a was dried in vacuum at 50-60° C. for 4-6 hours to afford the title compound.

### Example 15: Preparation of Crystalline Ibrutinib Form D1

1.0 g of ibrutinib was charged into the round bottom flask at 25-30° C. 40 mL of methanol was charged into the flask and the contents were heated to 50-60° C. The solution was made particle free and cooled to 2-10° C. In a separate flask, 40 mL of demineralized water was taken and cooled to 2-10° C. To this flask, the above cooled methanolic solution of ibrutinib was added slowly at 2-10° C. After the completion of the addition, the contents of the flask were maintained for 2-3 hours at 5-15° C. and filtered using vacuum pump. The obtained material was dried in vacuum at 45-55° C. for 4-6 hours to afford the title compound.

### Example 16: Preparation of Crystalline Ibrutinib Form D9

5.0 g of ibrutinib was charged into the round bottom flask at 25-30° C. 200 mL of methanol was charged into the flask and the contents were heated to 50-60° C. The solution was made particle free and cooled to 20-30° C. The contents of the flask were maintained for 20-24 hours at 20-30° C. and filtered. The obtained material was suck dried under vacuum at 25-30° C. for 30-60 minutes to afford the title compound.

### Example 17: Preparation of Crystalline Ibrutinib Form D9

70 mg of ibrutinib was taken into the well of high throughput screening instrument at 25-30° C. 0.15 mL of butanol and heptane (1:1) was added to the above well at the same temperature. The contents were vortexed at 25° C. for 26 hours. The compound was isolated by evaporation at 50° C. followed by nitrogen purging to obtain the title compound.

### Example 18: Preparation of Crystalline Ibrutinib Form D10

1.5 g of ibrutinib was taken into the well of high throughput screening instrument at 25-30° C. 3.0 mL of 1, 2-dimethoxy ethane was added to the above well at the same temperature. The well was cooled to 5° C. and vortexed at 5° C. for 13 hours. The contents of the well were filtered at 20-30° C. and dried under vacuum at 60° C. for 2-6 hours to obtain the title compound.

### Example 19: Preparation of Crystalline Ibrutinib Form D11

1.5 g of ibrutinib was taken into the well of high throughput screening instrument at 25-30° C. 3.0 mL of anisole was added to the above well at the same temperature. The well was cooled to 5° C. and vortexed at 5° C. for 13 hours. The contents of the well were filtered at 20-30° C. to obtain the title compound.

### Example 20: Preparation of Crystalline Ibrutinib Form D12

1.5 g of ibrutinib was taken into the well of high throughput screening instrument at 25-30° C. 3.0 mL of dimethyl carbonate was added to the above well at the same temperature. The well was cooled to 5° C. and vortexed at 5° C. for 13 hours. The contents of the well were filtered at 20-30° C. to obtain the title compound.

### Example 21: Preparation of Crystalline Ibrutinib Form D13

1.5 g of ibrutinib was taken into the well of high throughput screening instrument at 25-30° C. 3.0 mL of 1, 4-dioxane was added to the above well at the same temperature. The well was cooled to 5° C. and vortexed at 5° C. for 13 hours. The contents of the well were filtered at 20-30° C. to obtain the title compound.

### Example 22: Preparation of Crystalline Ibrutinib Form D1

30.0 g of ibrutinib was taken in 990 mL of methanol in a flask and heated to 50-60° C. to form a solution. The solution was added to a reactor charged with 1200 mL of demineralized water and 3.0 g of seed material of Form D1, maintained at a temperature of about 2-5° C. After the completion of the addition, the contents of the reactor were maintained at 0-5° C. for 15-20 hours. The reaction mass was filtered in a pre-cooled ANFD (Jacket temperature: 0° C. to 5° C.) under mild nitrogen pressure with simultaneous high vacuum. The material was dried in vacuum tray dryer at 25-30° C. for 8-9 hours to obtain the title compound.

1. Crystalline form D1 of Ibrutinib characterized by an X-ray Powder Diffraction Pattern (PXRD) having peaks at about 10.11, 11.45, 18.47 and 20.89±0.20 degrees 2θ.

2. Crystalline form D1 of Ibrutinib of claim 1, further characterized by PXRD pattern having additional peaks located at about 5.04, 10.79, 23.10 and 26.60±0.20 degrees 2θ.

3. Crystalline form D1 of Ibrutinib characterized by an X-ray Powder Diffraction Pattern (PXRD) as shown FIG. 18.

4. A process for preparing crystalline Form D1 of Ibrutinib, comprising the steps of:
a. obtaining a solution of ibrutinib in an alcohol;
b. combining the solution of step a) with an anti-solvent; and
c. isolating crystalline form D1 of Ibrutinib.

5. The process of claim 4 wherein, the alcohol is selected from methanol, ethanol, propanol, isopropanol, butanol, iso-butanol and pentanol.

6. The process of claim 4 wherein, the anti-solvent is water.
